(19) United States
(12) Patent Application Publication
(10) Pub. No.: US 2021/0149482 A1
(43) Pub. Date: May 20, 2021

(54) TRAINING SYSTEM AND METHOD UTILIZING A GAZE-TRACKING SYSTEM

(71) Applicant: Benny Council, New Orleans, LA (US)

(72) Inventor: Benny Council, New Orleans, LA (US)

(21) Appl. No.: 16/685,635

(22) Filed: Nov. 15, 2019

(51) Int. Cl. G06F 3/01; G06T 7/246; H04N 5/272; A63B 69/00; G02C 7/04; A42B 3/30

(52) U.S. Cl. CPC G06F 3/013; G06T 7/246; H04N 5/272; G06T 2207/30221; G02C 7/04; A42B 3/30; G06T 2207/10016; A63B 69/002

(57) ABSTRACT

A training system and method utilizing a gaze-tracking system are provided. An optical sensor and a scene camera are installed on a headgear, such as a football helmet, worn by a user. The optical sensor tracks eye movements of the user while wearing the headgear, and the scene camera is directed in a forward-facing direction to record the field of view of the user. Video of the field of view is recorded while the user's eye movements are simultaneously tracked in real time during a training exercise. The point of gaze of the user is then graphically superimposed onto the video. Thus, the video allows a coach to evaluate the performance of the user based on the user's visual focal point throughout the training exercise. The system may be used to train a quarterback in quickly and accurately assessing a play and delivering a ball to a receiver.

### FIELD OF THE DISCLOSURE

The present invention refers generally to a training system and method that utilizes a gaze-tracking system to simultaneously display the user's field of view and point of gaze in real time.

### BACKGROUND

There are numerous situations that require accurate and precise focus of the human eye. For instance, in a military setting, a soldier must have accurate vision when acquiring the location of a target in order to ensure his safety and the safety of other soldiers. In the medical field, a surgeon must be able to see precisely where each successive action should be taken in order to ensure a patient has received the best medical care. In many sports, athletes must be able to quickly and accurately make a visual determination of the most advantageous action the athlete may take to aid himself or his team in winning a game. For instance, in the sport of American football, a quarterback of a team possessing the ball must be able to quickly and accurately determine where and how the ball should be distributed to other players. In other sports, such as baseball, a player must be able to visually track a ball, either out of a pitcher's hand to hit a ball or off of a batter's bat to field a ball that has been hit in play.

In American football, specifically, a quarterback must be able to quickly and accurately scan the field to determine which receivers are open and which of those receivers may be in the most advantageous position to receive a pass from the quarterback. In this setting, it is essential that quarterbacks consistently practice timing in the passing game, including quick and accurate throws, in a variety of in-game scenarios in order to improve reflexes, timing, and decision-making when assessing where to pass the ball. A quarterback often must go through a progression of scanning different areas of the field of play to determine which receivers are open, which requires quickly shifting visual focus to different areas of the field, as quarterbacks have only a limited amount of time in which to throw the ball on a given play. Additionally, while deciding where to pass the ball on a given play, it is essential for a quarterback to recognize when opposing players are rushing the quarterback so that he may react accordingly in an effort to avoid being tackled by opposing players. When a quarterback repeatedly practices passing the ball to different receivers while also avoiding opposing players, coaches are able to evaluate the quarterback to make a determination as to how the quarterback will perform during a game against an opposing team. If a quarterback has slower reflexes or is less accurate than another quarterback on the same team, then it is optimal for a coach to place the quarterback who performs better in practice to play in live games against opposing teams. In addition, coaches may generally want to work on improving the passing skills of all quarterbacks on the team.

Part of the regiment for improving a quarterback's passing skill may involve improving the quarterback's ocular reflexes and accuracy. Being able to determine if a quarterback can visually locate the most advantageous receiver on a given play quickly and accurately in order to deliver the ball on time is critical for evaluating a quarterback's performance, as well as for evaluating a quarterback's capacity to improve his ocular reflexes. In order to evaluate a quarterback generally and/or a quarterback's capacity for improvement, it may be beneficial to know exactly where the eyes of the quarterback are focusing at any given moment during play. If it can be determined what a quarterback is seeing on the field at any given moment during play, a quarterback's play can be effectively evaluated, as well as a quarterback's capacity for applying coaching techniques from the coaching staff pertaining to where the quarterback should be focusing his line of sight during play. In this regard, quarterbacks are typically evaluated primarily subjectively through repetitive practicing of various designed passing plays. Such subjective evaluation may not provide a coaching staff sufficient information to make the most informed decisions possible regarding playing time for specific players. In addition, subjective evaluation may not give a coaching staff the best information regarding a quarterback's capacity for improvement.

Accordingly, there is a need in the art for a training system and method that can be used to evaluate a user's performance in terms of ocular reflexes. Further, there is a need in the art for a training system and method for evaluating the performance of athletes generally and, specifically, for evaluating the performance of a quarterback's in terms of visual progression in scanning a field to determine the most advantageous way of distributing the ball as quickly as possible.

### SUMMARY

In one aspect, a training system and method, which may be utilized as an athletic training system, are provided. The system and method track a user's eye movements in real time and superimpose the user's point of gaze onto streaming video data captured by a scene camera that captures an area of a field of view of the user. The system comprises a headgear, which may be an athletic helmet, such as a helmet used in American football, having an optical sensor and a scene camera secured to the headgear. The optical sensor is adapted to track eye movement and is positioned to track the eye movements of the user when the user is donning the headgear. The optical sensor may include one or more sensors working independently or in combination with each other to track eye movements. The scene camera is positioned facing in a forward direction from the user's head and is configured to capture an area of the field of view of the user when the user is donning the headgear. The system further comprises a display screen configured to display video data generated by the scene camera and a data processing unit in communication with the optical sensor, the scene camera, and the display screen. The system is configured to continuously determine a point of gaze of the user based on the eye movements of the user and to continuously display, on the display screen, the point of gaze superimposed onto streaming video data captured by the scene camera in real time.

To use the system, a user, who may be an athlete using the system for athletic training, dons the headgear and secures the headgear in a fixed position on the user's head so that the scene camera is positioned facing in a forward direction from the user's head and the optical sensor is placed in a position relative to the user's eyes to track the user's eye movements. Once the headgear is appropriately donned by the user, the user then engages in a training exercise by physically simulating real-world conditions that the user is likely to experience. For instance, the training exercise may be an athletic training play, such as a play run by a team playing American football. The user may run a variety of plays to simulate real-world conditions that the user is likely to experience throughout a football game. While engaging in the training exercise, video of the field of view of the user is recorded by the forward-facing scene camera while simultaneously tracking the user's eye movements in real time. In addition, while engaging in the training exercise, the display screen graphically displays the user's point of gaze continuously superimposed onto the recorded video over a period of time that the system is activated during the simulated exercise. Thus, the present system provides a continuous visual representation of the precise location of where a user is looking within the user's field of view over a period of time.

In a preferred embodiment, the present system and method may be utilized in training athletes and may be particularly advantageous in training athletes that play the quarterback position in American football. Thus, the system provides a football coach with accurate information regarding the quarterback's point of gaze during practice plays, which allows the coach to effectively evaluate the quarterback's performance in terms of how the quarterback visually scans different areas of the field of play to identify open receivers or opposing players on the field. Thus, the system may allow the coach to evaluate if the quarterback is quickly making correct decisions regarding passing the ball to a poorly guarded receiver or to a heavily guarded receiver that may increase the risk of interception of the ball by an opposing player. The system may also allow the coach to evaluate pre-snap reads by the quarterback of a defensive formation by seeing how the quarterback is identifying specific players in the formation.

The present system may also provide valuable information in evaluating a quarterback's performance in a variety of other ways. For instance, the system may allow a coach to determine which direction a player is turning his head after a play is initiated. If the play requires the player to be focusing his area of view down the field but the player continuously turns his head to focus on the peripheral areas of view, the coach can use the present system to correct this behavior. Further, the system may allow a coach to recognize exactly where the player is focusing his point of gaze in relation to players down the field. For example, if a particular play call requires offensive players to run down the field and cross the field in horizontal or diagonal passing routes, the coach can determine if the quarterback is able to focus his gaze on which player represents the optimal player to pass the ball to.

Additionally, the present system may allow a coach to evaluate the precision with which the quarterback is able to locate a throw. For example, if a play requires an offensive player to run downfield and also requires the quarterback to throw the ball ahead of the offensive player in an effort to precisely locate the throw downfield for the offensive player to receive the ball, the coach can determine if the point of gaze of the quarterback is where the quarterback should be looking when assessing where to place the ball for that particular play. The present system allows for uninterrupted viewing of the exact point of gaze of a player as it relates to the area of view of the player in real time with low latency.

Although the present system and method are particularly advantageous for coaches in analyzing point of gaze of a football player in real time, the present system and method may also be used in other applications, such as medical procedures and military or police training applications.

### DESCRIPTION OF THE DRAWINGS

These and other features, aspects, and advantages of the present invention will become better understood with regard to the following description, appended claims, and accompanying drawings where:

FIG. 1 shows a gaze-tracking system in use by a user in accordance with the present disclosure.

FIG. 2 shows a perspective view of a headgear for use with the present gaze-tracking system in accordance with the present disclosure.

FIG. 3 shows a front elevation view of a contact lens for use with the present gaze-tracking system in accordance with the present disclosure.

FIG. 4 shows a schematic diagram of the contact lens shown in FIG. 3 in accordance with the present disclosure.

FIG. 5 shows a partial cross-sectional view of the contact lens shown in FIG. 3 for use with the present gaze-tracking system in accordance with the present disclosure.

FIG. 6 shows an illustrative embodiment for a sensor for use with the present gaze-tracking system in accordance with the present disclosure.

FIG. 7 shows a gaze-tracking system in use by a user in accordance with the present disclosure.

FIG. 8 shows a schematic diagram of components of the present gaze-tracking system in accordance with the present disclosure.

FIG. 9 shows a schematic diagram of components of the present gaze-tracking system in accordance with the present disclosure.

### DETAILED DESCRIPTION

In the Summary above and in this Detailed Description, and the claims below, and in the accompanying drawings, reference is made to particular features, including systems and method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, ingredients, steps, etc. are optionally present. For example, an article “comprising” components A, B, and C can contain only components A, B, and C, or can contain not only components A, B, and C, but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “vector quantity data” and grammatical equivalents thereof are used herein to mean data representing measurements of vectors of infrared light being reflected off the cornea of a user and measured from two points with one point representing the center of the pupil of the user and the other point representing a fixed location on the cornea of the user. Such vector quantity data is determined as a quantity having direction as well as magnitude, especially as determining the position of one point in space relative to another, said two points being the above-mentioned pupil center and fixed location against the cornea.

Eye tracking is the process of measuring the point of gaze of a subject's eye. Point of gaze is, generally, where the user is looking. Specifically, point of gaze focuses on a particular point in the user's area of view that is the focal point of the user's vision. The point of gaze may be determined as a totality of fixational eye movements, saccadic eye movements, and smooth pursuit eye movements in a given period of time and space. Eye tracking, or gaze tracking, is a method of measuring these eye movements over a period of time.

In one aspect, a gaze-tracking system and a method of training utilizing the system are provided. The present system and method are particularly advantageous for use in athletic training for analyzing the point of gaze (hereinafter “POG”) of an athlete, such as a player in American football. However, the present system and method may be used in other applications, such as medical procedures or training simulators in civilian or military settings. The system and method track eye movements of a user 101 in real time and superimposes the user's point of gaze onto streaming video data captured by a scene camera 104 that captures an area of a field of view of the user 101. The system comprises a headgear 105, which may be an athletic helmet, such as a helmet used in American football, having an optical sensor 103 and a scene camera 104 secured to the headgear 105. The optical sensor 103 is adapted to track eye movement and is positioned to track the eye movements of the user 101 when the user is donning the headgear 105. The scene camera 104 is positioned facing in a forward direction from the user's head 101 and is configured to capture an area of the field of view of the user 101 when the user is donning the headgear 105. The system further comprises a display screen 702 configured to display video data generated by the scene camera 104 and a data processing unit 701 in communication with the optical sensor 103, the scene camera 104, and the display screen 702. The system is configured to continuously determine a point of gaze 401 of the user 101 based on the eye movements of the user and to continuously display, on the display screen 702, the point of gaze 401 superimposed onto streaming video data captured by the scene camera 104 in real time.

To use the system, a user 101, who may be an athlete using the system of athletic training, dons the headgear 105 and secures the headgear 105 in a fixed position on the user's head 101 so that the scene camera 104 is positioned facing in a forward direction from the user's head and the optical sensor 103 is placed in a position relative to the user's eyes to track the user's eye movements. Once the headgear 105 is appropriately donned by the user 101, the user then engages in a training exercise by physically simulating real-world conditions that the user is likely to experience. In a preferred embodiment, the training exercise may be an athletic training play, such as a play run by a team playing American football, which may be run by the user 101 along with other players on the team. The user (and additionally, in the case of certain team sports, the user's teammates) may run a variety of plays to simulate real-world conditions that the user is likely to experience throughout a football game. While physically engaging in the training exercise, video of the field of view of the user is recorded by the forward-facing scene camera 104 while simultaneously tracking the user's eye movements in real time. For instance, when running a football play, an athlete's eye movements are tracked to see where on the field the athlete 101 is looking. This includes players on the athlete's own team, as well as players simulating the players on an opposing team. In addition, while engaging in the training exercise, the display screen 702 graphically displays the user's point of gaze 401 continuously superimposed onto the recorded video over a period of time that the system is activated during the simulated exercise. Thus, the present system provides a continuous visual representation of the precise location of where a user is looking within the user's field of view over a period of time. As such, a coach may evaluate a quarterback 101 by viewing the quarterback's point of gaze 401 on the display screen 702 to see which players the quarterback is focusing on, including how long the quarterback focuses on a particular player or area of the field, as well as how quickly the quarterback scans the field and focuses on other players in succession.

Accordingly, the present system may provide a football coach with accurate information regarding the quarterback's point of gaze during practice plays, which allows the coach to effectively evaluate the quarterback's performance in terms of how the quarterback visually scans different areas of the field of play to identify open receivers or opposing players on the field. Thus, the system may allow the coach to evaluate if the quarterback is quickly making correct decisions regarding passing the ball to a player who may be heavily guarded or an available player not heavily guarded yet at risk of interception of the ball by an opposing player. The system may also allow the coach to evaluate pre-snap reads by the quarterback of a defensive formation by seeing how the quarterback is identifying specific players in the formation.

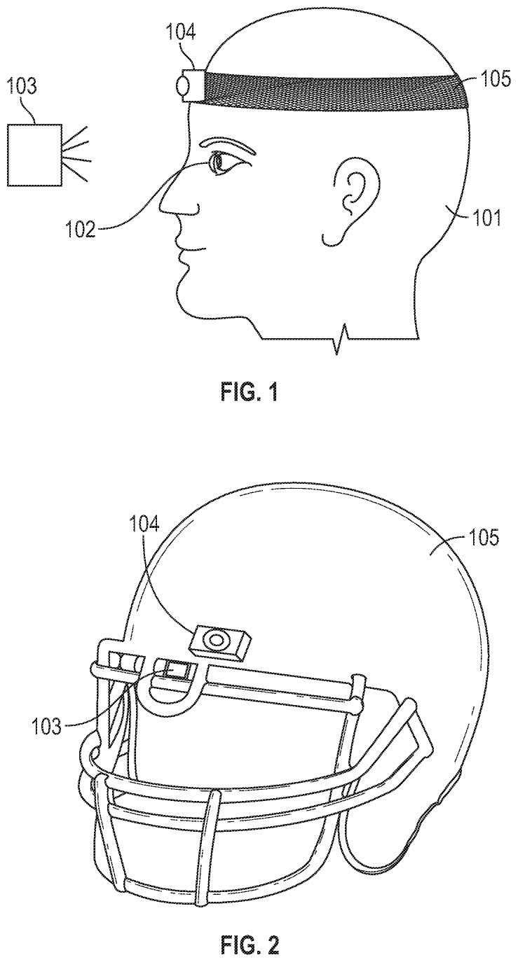

Turning now to the drawings, FIGS. 1-9 illustrate preferred embodiments of the present gaze-tracking system that may be utilized in training exercises, including athletic training. The system may preferably utilize a combination of sensors, cameras, and data processing units utilizing wireless data transmission to display the exact point of gaze of a user of the system. FIG. 1 illustrates a user 101 donning a headgear 105, which in a simple form may be a headband, having a scene camera 104 secured to the headgear. FIG. 1 also illustrates the user 101 wearing an optional first sensor 102, which may be in the form of a contact lens, on the user's eye. The first sensor 102 may function as a point of reference for a second sensor 103. The first and second sensors are optical sensors that may be used separately or in combination to track the user's eye movements.

FIG. 2 illustrates a preferred embodiment of a headgear 105 as an athletic helmet, which in this case is a helmet type typically worn by American football players, to be worn on the head of the user 101. The helmet is configured to secure a scene camera 104 to the helmet on the frontal exterior portion of the helmet. The scene camera 104 is configured to capture at least a portion of the central and peripheral areas of view of the user 101. The helmet is further configured to secure a second sensor 103, which is an optical sensor adapted to track eye movement and specifically positioned relative to at least one of the user's eyes to track the eye movements of the user 101 while donning the headgear 105. Other non-exhaustive embodiments of the headgear 105 may utilize various types of headgear 105 that conform to a particular use of the user 101 while still maintaining the purpose of the system. For example, use of the system as a military training system may require the headgear 105 to be a personal armor helmet or other type of military headgear specifically used to protect the head during combat. In a medical training scenario, the headgear 105 may be a surgical cap used to cover the head hair of a surgeon or a specifically designed headgear for such use. In other embodiments, the present system and method may be utilized in other sports, such as baseball. In this case, the helmet 105 may be a baseball helmet similar to helmets commonly worn by baseball players.

FIG. 2 further illustrates one embodiment of the placement of the second sensor 103 relative to the point of reference defined by the first sensor 102. The second sensor 103 may be generally affixed to the headgear 105 and positioned anterior to the cephalic region of the user 101 while remaining out of the direct line of sight of the user 101. The placement of the second sensor 103 as seen in FIG. 2 allows for accurate gathering of data while remaining outside the area of view of the user 101 so as to not interfere with the line of sight of the user 101. The placement of the second sensor 103 in the position as illustrated additionally serves the purpose of remaining close enough to the first sensor 102 that a magnetic field sensor 603 may be able to accurately measure the changes in polarization emitted from magnetic material 502 embedded within the first sensor 102. The placement of the second sensor 103 in this position may also serve the purpose of accurate data gathering by method of infrared corneal reflection eye tracking. The second sensor 103 may be placed in such a position that an infrared illuminator 601 may cast infrared light against the eye of the user 101 when such infrared light emitted from the sources of light surrounding the user 101 is insufficient for projecting infrared corneal reflections from the eye of the user 101. Additionally, such placement allows an infrared vector camera 602 to be placed in such a position that infrared corneal reflections may be accurately measured and such data collected for transmission to a data processing unit 701.

The scene camera 104 is a camera capable of recording at least a portion of the central and peripheral areas of view of the user 101. The scene camera 104 is preferably positioned close enough to the line of sight of the user 101 to generally represent what the user 101 is seeing without interfering with the vision of the user 101. In a preferred embodiment, the scene camera 104 is an action camera capable of recording dynamic video in real time at varying resolutions corresponding to a predetermined frame rate for maximum resolution at the set frame rate.

In a preferred embodiment, the scene camera 104 may be capable of recording continuous video at a resolution of 1280 pixels by 960 pixels recorded at 24 frames per second. If the user 101 desires to increase the frame rate of video recording to 30 frames per second, the video resolution may need to be adjusted to 960 pixels by 720 pixels. In a preferred embodiment, the recording dimensions of the scene camera 104 may allow for at least an 80° horizontal and 60° vertical area of recording in order to accurately represent the front-facing scene of the user 101. Both recording options allow for high-definition video recording giving clear representation of POG data to an observer of the data. The scene camera 104 may also be configured to automatically adjust settings for maintaining high sensitivity in conditions with low light. The scene camera 104 is configured to transmit the streaming feed of video data to a data processing unit 701 so that the video may be integrated with data originating from the optional first sensor 102 and the second sensor 103 with the ultimate destination for each respective portion of data to be synchronized into one stream of data representing the POG of the user 101.

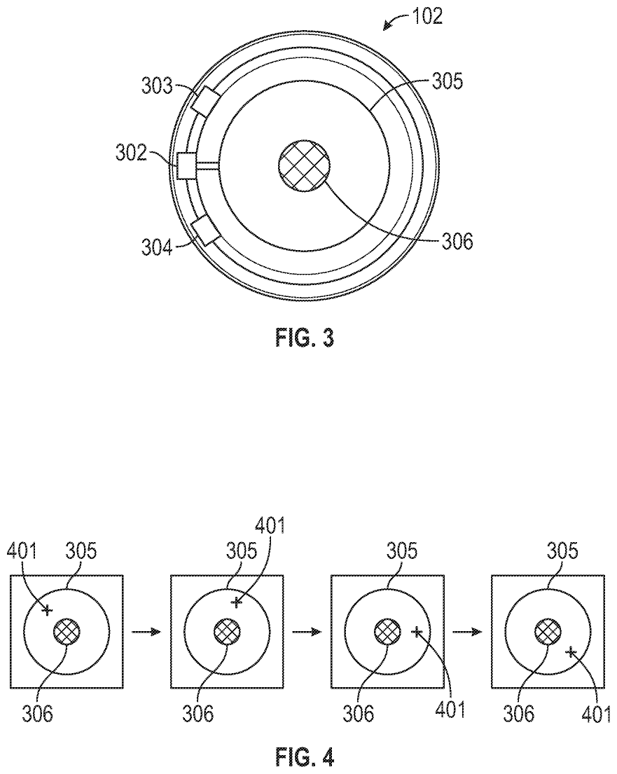

FIG. 3 illustrates a preferred embodiment of the optional first sensor 102, which is preferably in the form of a contact lens generally placed in a fixed position on at least one eye of the user 101 and serving as a point of reference. FIG. 3 also illustrates various components that may be contained within the first sensor 102, which components may preferably include: an integrated circuit 303, a power source 302, a transmitter 304, and a contact lens camera 305 surrounding the pupil 306 of the user 101.

The contact lens camera 305 is preferably a digital camera capable of recording a series of thin-film images corresponding to the motion of the eye of the user 101 in real time. This camera 305 may be particularly advantageous for capturing a series of fixational eye movements, saccadic eye movements, and/or smooth pursuit eye movements while remaining small enough to be contained within a portion of the contact lens as to not interfere with the area of view of the user 101. FIG. 4 shows an illustrative sequence of the contact lens camera 305 within the first sensor 102 using thin-film images to capture the gaze point 401 of the user 101. The camera 305 may be further configured to capture vector quantity data representing the POG of the user 101. As such, the camera 305 may be in communication with the integrated circuit 303. The camera 305 may transmit thin-film image data as well as vector quantity data to the integrated circuit 303 for the purpose of storing and relaying input data originating from the camera 305 to the transmitter 304. The camera 305 is connected to and powered by the power source 302 as a means of supplying electrical power from the power source 302 to the camera 305. The connection between the camera 305 and the power source 302 may be by direct contact through a filament or any other similar conductive means. Connection may also be achieved by radio wave, electromagnetic induction, or electromagnetic field resonance. Such wireless methods of supplying the contact lens camera 305 with electrical power may be achieved through communication between the camera 305 and the transmitter 304, which may also be connected to the power source 302 via filament or similar conductive means.

In a preferred embodiment, the power source 302 may comprise a hybrid supercapacitor capable of electrostatic and electrochemical charge storage. The hybrid supercapacitor may operate under atomic layer deposition in combination with chemical vapor deposition to coat transition metal materials onto a variety of substrates as a means of charge storage. This may provide the advantage of high energy density, high stability, and long operation lifetime. The power source 302 may also comprise a supercapacitor or a pseudocapacitor.

In a preferred embodiment, the transmitter 304 may comprise a wireless chipset. The wireless chipset is capable of data transmission to configured external receivers without the need for physically attached transmission components between respective parts. The data transmission may be achieved by infrared wave transmission, radio wave, electromagnetic induction, or electromagnetic field resonance. This may provide the advantage of consistent and accurate data transmission with low latency and low signal interference. The transmitter 304 may also comprise an internal antenna to aid with the transmission of the wireless stream of data in an effort to further reduce transmission latency and interference.

In a preferred embodiment, the integrated circuit 303 may comprise a microcontroller in communication with the contact lens camera 305, the power source 302, and the transmitter 304. The microcontroller may comprise a central processing unit with semiconductor memory elements further comprising memory cells capable of random-access memory, read-only memory, and flash memory. The integrated circuit 303 is configured to receive input data from the camera 305 but may additionally be configured to receive input data from components of the second sensor in order to reduce latency and signal interference. As an alternative to the microcontroller, the integrated circuit 303 may also comprise a microprocessor, field-programmable gate array, or system on a chip. The integrated circuit 303 may also comprise at least one inductive sensor connected to a conditioning electronic circuit for the purpose of creating a search coil magnetometer. Such a magnetometer may use electromagnetic induction using current generated from the power source 302 to generate electrical currents within the search coil as a means of measuring alternating magnetic fields generated by the magnetic material 502 embedded within the first sensor 102. These electrical currents generate polarity and amplitude that vary with the direction, angular displacement, and torsional rotation of the eye of the user 101. These values may be measured by the at least one inductive sensor and communicated to the transmitter 304. This allows for components within the first sensor 102 to measure data related to the magnetic field generated by other components within the first sensor 102 and synchronize with other data representing the POG of the user 101.

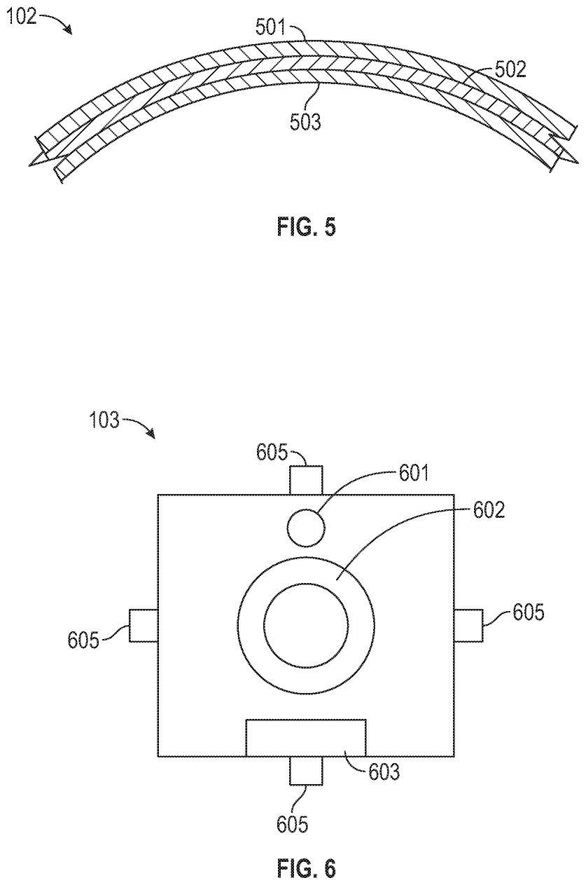

FIG. 5 illustrates a cross-section of a preferred embodiment of the first sensor 102 showing the magnetic material 502 embedded within the first sensor 102. The magnetic material 502 may be layered between an exterior portion of the first sensor 501 and an interior portion of the first sensor 503 that rests against the eye of the user 101. The first sensor 102 may be embedded with magnetic material to create a magnetic field emitting from the first sensor from which the polarization may be measured by the second sensor 103. As the eye of the user 101 moves, the first sensor 102 moves with the eye and creates subtle changes in the polarization of the magnetic field emitted by the first sensor 102. These changes in polarization may be measured by the second sensor 103 and processed by the data processing unit 701 into data representing the POG of the user 101. This process yields sensitive and accurate recordings of the eye movements of the user 101 when associated with a first sensor 102 that is capable of fitting firmly against the eye of the user 101. This system of gathering magnetic polarization data representing the POG of the user 101 may also be used as a means for supplementing POG data gathered through infrared corneal reflection tracking, as well as data gathered by means of the thin-film camera 305 within the first sensor. The magnetic material 502 that may be embedded within the first sensor 102 may include, but is not limited to, iron, nickel, cobalt, alnico alloy, ferrite, neodymium alloy, copper, and manganese. The magnetic material 502 is preferably embedded in the first sensor 102 in amounts capable of creating a magnetic field by which polarization can be measured but not in amounts great enough to interfere with the vision of the user 101 wearing the lens.

FIG. 6 illustrates a preferred embodiment of the second sensor 103 as a collection of components that may be secured in a suitable position relative to at least one eye of the user 101 for the purpose of tracking the user's eye movements by collecting data representing the POG of the user 101. The second sensor 103 is an optical sensor that preferably measures infrared corneal light reflections to track eye movement. The second sensor 103 may be used for directly tracking eye movement and/or for measuring data originating from the point of reference defined by the first sensor 102. Thus, in a preferred embodiment, the second sensor 103 may be utilized independently or in combination with the first sensor 102 to track user eye movement. FIG. 6 illustrates various components of the second sensor 103, which may preferably comprise: an infrared illuminator 601, an infrared vector camera 602, a magnetic field sensor 603, and mounting points 605.

In a preferred embodiment, the infrared illuminator 601 uses a light emitting diode (LED), which may comprise gallium arsenide or aluminum gallium arsenide, and which may emit invisible infrared light around 760 nanometers under a voltage of 1.4 volts. The second sensor 103 may also be equipped with more than one infrared illuminator 601, which may activate if the source of infrared light is inadequate to produce sufficient corneal reflections. The location of each additional infrared illuminator 601 may be varied. In a preferred embodiment, the infrared vector camera 602 is a camera directed to capture infrared light reflections against the eye of the user 101. The infrared vector camera 602 may capture infrared light reflections in the light spectrum of 700-1000 nanometers and may be outfitted with long-pass optical filters for the purpose of filtering visible light out of the spectrum of light to be measured. In another embodiment, the second sensor 103 may use more than one infrared vector camera 602 synchronized with the first infrared vector camera 602 for the purpose of acquiring stereo images simultaneously. Preferably, the infrared vector camera 602 is capable of capturing at least 30 images per second using high-speed shutters and progressive scan in an effort to reduce motion blur as a natural result of saccadic movements. The infrared vector camera 602 may also be equipped with pan-tilt-zoom features, which allow the camera 602 to focus on and automatically track a designated, specific portion of the eye of the user 101 to optimally capture infrared corneal reflections.

In a preferred embodiment utilizing the optional first sensor 102, the magnetic field sensor 603 comprises a search coil vector magnetometer measuring at least one component of the magnetic field produced by the first sensor 102. The magnetic field sensor 603 may comprise at least one orthogonal inductive sensor that operates in a similar manner as the search coil magnetometer contained within the integrated circuit 303 and may operate as a secondary means of measuring additional polarization information generated by the magnetic field. The magnetic sensor 603 may also be in communication with the transmitter 304 for the purpose of gathering POG data collected by the integrated circuit 303. This mechanism serves as an additional safeguard for storage and transmission of data originating from the integrated circuit 303 in an effort to reduce the effect of unwanted signal interference between the first sensor 102 and the data processing unit 701.

To secure the second optical sensor 103 to the headgear 105, there may preferably be a plurality of attachment points 605 positioned around a perimeter of the second sensor 103. The attachment points 605 may provide securing structures by which the second sensor 103 may be mounted onto the headgear 105 in an appropriate position relative to the eyes of the user 101 and/or the point of reference provided by the first sensor 102 for accurate data collection. These attachment points 605 may be detachable from the second sensor 103 if one or more attachment points 605 would interfere with proper adhesion to a respective mounting point. The attachment points 605 may attach to a desired mounting point by any suitable means, including, but not limited to, a malleable curved plastic piece, a mounting plate with securing screws, or adhesive material.

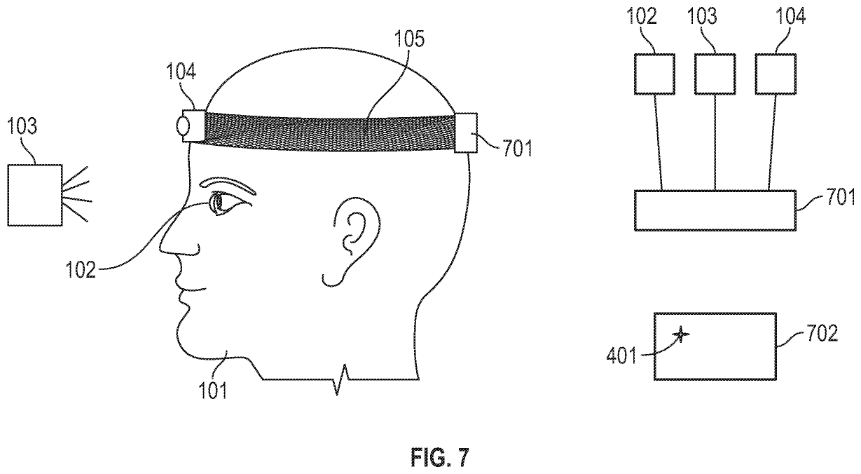

FIG. 7 illustrates a preferred embodiment of the present system that includes a data processing unit 701 secured to the headgear 105. FIG. 7 further shows the present system schematically, including a representation of communications between the first sensor 102, the second sensor 103, the scene camera 104, the data processing unit 701, and the image viewer 702, which includes the display screen for viewing the user's POG superimposed onto the streaming video. FIG. 7 depicts one illustrative embodiment of the present system wherein the data processing unit 701 may preferably be secured to a back end of the headgear 105 resting against a posterior cephalic region of the user 101. This positioning of the data processing unit 701 may serve the purpose of retaining the data processing unit 701 within an acceptable range for accurate and consistent wireless data transmission between the components of the system to the data processing unit 701. In another embodiment, the data processing unit 701 may be placed in any other suitable area relative to the user 101 that provides consistent and accurate data transmission.

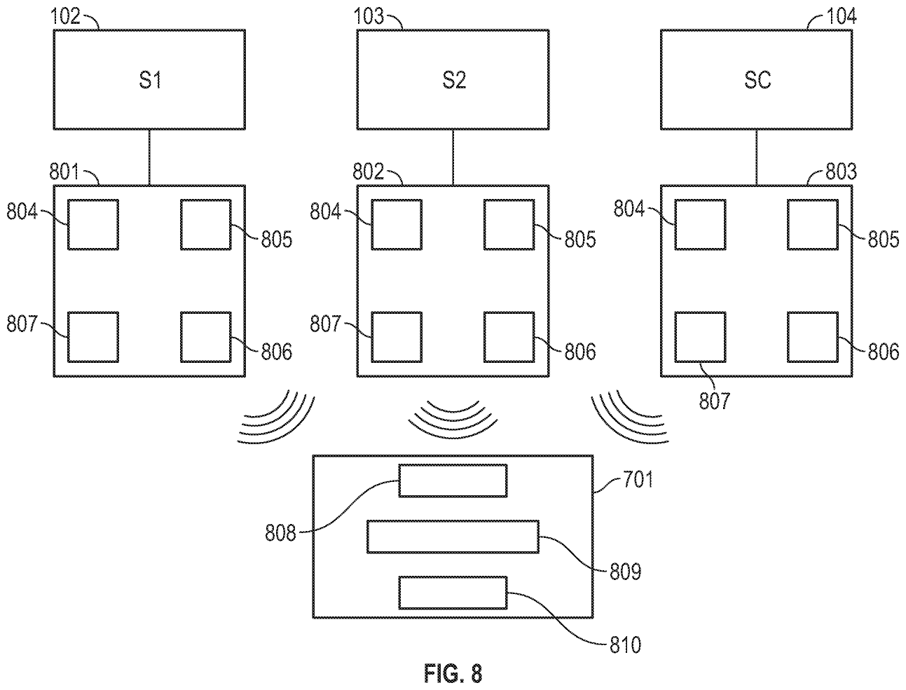

In a preferred embodiment, the data processing unit 701 comprises a central processing unit 810, as shown in FIG. 8. The central processing unit 810 is preferably configured to receive at least the following: input vector quantity data and magnetic polarization data from the first sensor 102, input vector quantity data and magnetic polarization data from the second sensor 103, and streaming video data in real time from the scene camera 104. The central processing unit 810 may also be configured to receive data from any other sources that may be contained in alternative embodiments of the system. The central processing unit 810 may further comprise a non-transitory computer-readable medium coupled to the processing unit 810 and having a set of instructions stored thereon, which, when executed, synchronizes the input vector quantity data and the magnetic polarization data from the first sensor 102 with the input vector quantity data and the magnetic polarization data from the second sensor 103 into a singular quantitative data stream representing the point of gaze of the user 101. The computer-readable medium may have a further set of instructions stored thereon, which, when executed, converts the singular quantitative data stream into a graphical representation 401 of the point of gaze of the user 101. Finally, the computer-readable medium may have a finishing set of instructions stored thereon, which, when executed, superimposes the graphical representation 401 of the point of gaze of the user 101 over the streaming video data generated by the scene camera 104 in real time.

FIG. 8 further illustrates preferred components of the data processing unit 701, which may include, but are not limited to, a power source 808 used to supply electrical current to the data processing unit 701, the central processing unit 810, and a non-volatile data storage unit 809 used for storing input data received from various sources in the system.

In a preferred embodiment, the power source 808 may comprise an internal battery capable of being recharged. The rechargeable battery may preferably be a lithium-ion battery or a lithium-ion polymer battery. Such sources of power may provide the advantage of high energy density and low rate of discharge allowing for long periods of use by the user 101 before the system needs to be recharged. In an alternative embodiment, the power source 808 may comprise a hybrid supercapacitor capable of electrostatic and electrochemical charge or an ultracapacitor. In another embodiment, the power source 808 may combine the primary source of power with a betavoltaic battery to create a hybrid betavoltaic power source. This system may provide the added advantage of providing a trickle-charge to the power source used which may increase the energy capacity and overall lifespan of the system as a whole.

In a preferred embodiment, the non-volatile data storage unit 809 may comprise electrically erasable programmable read-only memory in the form of flash memory. After being processed into a single stream of data representing the POG of the user 101, the data may be transmitted to the non-volatile data storage unit for copying and preservation of the data before the data is directed to the process of distribution across the system. This may serve the purpose of storing POG data as the central processing unit 810 receives the data so that the data may be reviewed multiple times as opposed to only viewing the data as it occurs in real time.

FIG. 8 illustrates a series of nodes connected respectively to each of the optional first sensor 102, the second sensor 103, and the scene camera 104. These nodes may provide the primary means for wireless communication from the integral components of the system to the data processing unit 701, of which the totality of nodes creates a wireless sensor network. In this preferred embodiment, a first node 801 is connected to the first sensor 102, a second node 802 is connected to the second sensor 103, and a third node 803 is connected to the scene camera 104. The first 801, second 802, and third 803 nodes are in communication with the data processing unit 701. Each node is configured for transmitting data wirelessly to the data processing unit preferably with as little latency and interference in data transmission as possible. This may provide the advantage of reducing the possibility of wired connections between components being damaged during normal use of the system, as well as reduced weight on the head of the user 101 and increased range of mobility and freedom of placement choice of the system components. Further, the present system of three nodes for data transmission across the system may represent the minimum amount necessary for such data transmission, though other nodes may be incorporated into the system for enhanced data transmission. Such nodes may include an end node capable of transmitting data to a collection of system resources (i.e. a “cloud”).

Because the nodes all perform similar functions, the components of each node may generally be the same. Each node may comprise a transmitter 804, a microcontroller 805, an electronic circuit 806, and a power source 807. In one embodiment, the wireless sensor network comprises each of the first sensor 102, second sensor 103, and scene camera 104 being hardwired to each respective node via filament or other equivalent conductive means. Because each node is in close physical proximity to each respective sensor, having a hardwired connection between the respective components allows for secure and reliable data transmission from the data origin point to each respective node.

In one preferred embodiment, the transmitter 804 may comprise an internal antenna capable of transmitting input data received from the point of origin to the next destination, which, in the present case, would be the data processing unit 701. This form of data transmission is a reliable form of data transmission that results in accurate transmission with low latency. The transmitter 804 may also be in the form of a transceiver, which may be capable of both transmitting data to a secondary source as well as receiving input data from the secondary source. The transmitter 804 may also provide for a means to connect to an external antenna as a way of increasing the reliability of data transmission.

In a preferred embodiment, the microcontroller 805 may comprise components similar to the microcontroller contained in the integrated circuit 303, although the scale of components of microcontroller 805 need not be so reduced. The microcontroller 805 may comprise a central processing unit with semiconductor memory elements further comprising memory cells capable of random-access memory, read-only memory, and flash memory. These components may be configured for both receiving input data originating from each respective sensor or camera, storing such data for future transmission, and forwarding the data to the transmitter 804. A secondary purpose of the microcontroller 805 may be to function as a safeguard for controlling the functions of the respective sensor or camera to which the node is hardwired. This system may allow for increased reliability of the sensors used in the system should one of the sensors fail.

The electronic circuit 806 may provide a simple means for connectivity between the above-referenced components comprising the node. In a preferred embodiment, the electronic circuit 806 may comprise a hybrid circuit containing elements of both digital and analog circuits and may be configured to support the addition of optional components such as resistors, transistors, capacitors, inductors, or diodes. The power source 807 may comprise a capacitor. Similar to power source 302, in a preferred embodiment, the power source 807 may comprise a hybrid supercapacitor capable of electrostatic and electrochemical charge storage, which may provide a power source 807 with many of the same advantages of operation applicable to power source 302. Further, additional embodiments of the power source 807 may comprise a supercapacitor or a pseudocapacitor.

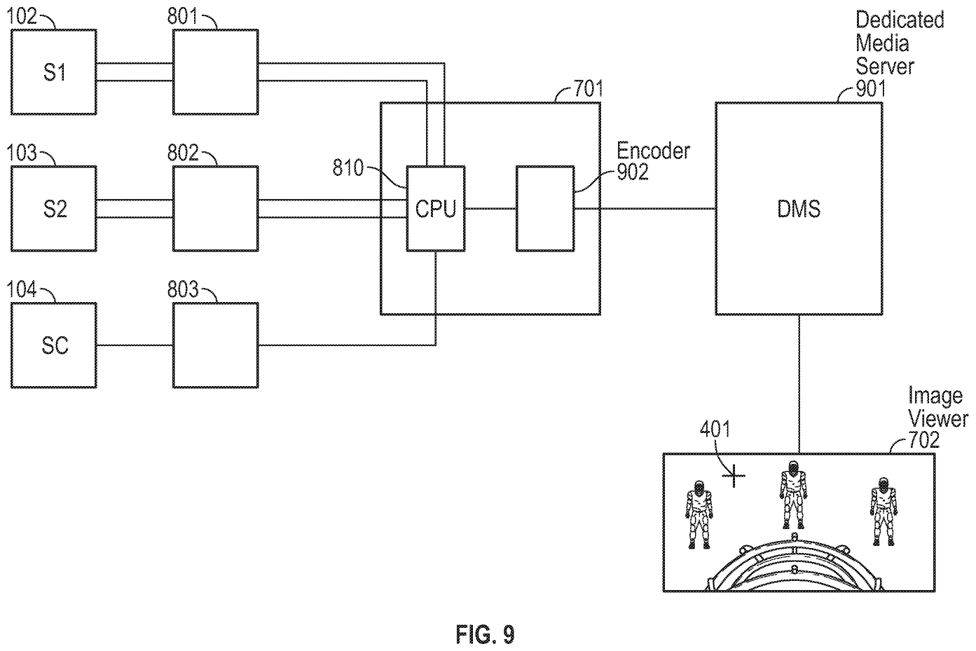

FIG. 9 shows a preferred embodiment of the system illustrating the dynamic interaction between the optional first sensor 102, the second sensor 103, the scene camera 104, the first node 801, the second node 802, and the third node 803 with the data processing unit 701 in further communication with a dedicated media server 901, and finally, the image viewer 702. In a preferred embodiment, all data representing the point of gaze of the user 101 converges within the central processing unit 810 of the data processing unit 701. Once synchronized and converted to a graphical representation 401 of the POG of the user 101, the data is superimposed over the stream of video images generated by the scene camera 104. This data feed may eventually be transmitted to a dedicated media server 901 with the final destination for the data being the image viewer 702 to be viewed by a coach or other observer for evaluation of the user's performance.

In a preferred embodiment, the transmission of data from the central processing unit 810 may begin with a process of encoding the data stream in real time. As such, the data processing unit 701 may preferably comprise an encoder 902 component. The encoder 902 may compress the size of the input data to a more manageable size for transmission to the server 901. This process may provide the advantage of high throughput as well as high efficiency with low latency. Further, the system may be further configured to reduce data payload size transmission should latency need to be reduced. In one preferred embodiment, the encoder 902 comprises a dedicated collection of hardware contained within the data processing unit 701. In another embodiment, the encoder 902 may comprise a non-transitory computer-readable medium coupled to the data processing unit 701 and having a set of instructions stored thereon, which, when executed, performs the encoding process without additional hardware components.

The encoded data may then be transmitted to the dedicated media server 901. The encoded data may preferably be transmitted by a communications protocol. In this case, the preferred protocol may preferably be Real-Time Messaging Protocol (“RTMP”). The data may be fractured into specified payload sizes and transferred to the dedicated server 901 over a secure connection. This method of data transfer delivers data streams smoothly, transmits as much information as the system can provide while providing reliable, continuous connections and low-latency communication. Other embodiments of data transfer processes that may be utilized are Hypertext Transfer Protocol, Real-Time Streaming Protocol, Session-Description Protocol, or any combination thereof.

The dedicated media server 901 may ingest the data payload originating from the encoder 902 through a communications protocol. The dedicated media server 901 may be contained within the data processing unit 701, within the image viewer 702, or may be a stand-alone unit. The server 901 processes the incoming data payload units (“bit rates”) and transform the data into the required medium for viewing using the image viewer 702. This may be accomplished by changing the data format, resolution, and frame rate, which may allow for automatic adaptation of the information to best conform with the conditions set by the viewer's network and playback conditions. The system allows the best possible viewing conditions with little buffering, fast start times, and quality viewing regardless of the connectivity condition.

The server 901 may allow for the format of the data to be changed according to the type of media viewer being used. This gives the viewer flexibility regarding viewing the incoming data stream. As such, a preferred embodiment of the image viewer 702 may include computer instructions conditioned to receive the data input stream from the dedicated media server and project such data to the viewer in a compatible manner. Therein, the image viewer 702 may be adapted for use as an application to display the POG of the user 101 against an existing dedicated viewing system, which may, for example, be a mobile phone, tablet, or computer. The system may also be configured to project POG data against a dedicated web-browser if the viewer does not desire to use a dedicated application. Such a system allows flexibility for the viewer to best configure the system for use with components already possessed by the viewer.

A method of training utilizing the present gaze-tracking system is also provided. The present method generally comprises the steps of providing a headgear 105 and a gaze-tracking system including certain components of the system secured to the headgear, which components include an optical sensor 103 and a scene camera 104; donning the headgear 105, by a user 101, who may preferably be an athlete when utilizing the system for athletic training; engaging in a training exercise by physically simulating real-world conditions, which may comprise running an athletic training play as the training exercise, such as a scripted play in the sport of American football to simulate in-game conditions that a player is likely to encounter; recording video with the scene camera 104 of the field of view of the user 101 while simultaneously tracking eye movements of the user in real time while the user is simulating real-world conditions for the training exercise; and graphically displaying, on the display screen 702, the user's point of gaze superimposed onto the recorded video over a period of time in which the gaze-tracking system is activated while the user is simulating real-world conditions. In a preferred embodiment, the training exercise being simulated may be a football play in which offensive players, such as wide receivers, tight ends, or running backs, run pass routes while defenders attempt to cover the offensive players. In other illustrative embodiments, the training exercise being simulated may be a simulated baseball game in which the user 101 is a batter attempting to hit a ball being thrown by a pitcher or a pitching machine to evaluate how the batter sees and visually tracks the ball as it moves toward the batter. In an alternative use involving baseball, the user 101 may be a fielder fielding fly balls to evaluate how the fielder tracks the ball off a batter's bat when the ball is hit. In other alternative embodiments, the user 101 may be a law enforcement officer or member of the military, and the system may be utilized to track the user's point of gaze in scanning his or her environment to identify potential threats. In any of these illustrative cases, the simulated training exercise is a real-world exercise that is filmed by the scene camera 104 while the user's eye movements are tracked. A “real-world” exercise or “real-world” conditions may generally refer to any physical environmental conditions that a user of the system is likely to experience when performing the task for which the user is training.

A viewer, such as a football coach, may then view a graphical representation of the point of gaze, which is generally the focal point of the user 101, continuously on the display screen 702 while the system is activated during the training exercise to see exactly where the user is focusing his vision within the user's field of view, which may allow the viewer to effectively evaluate the performance of the user 101 in carrying out the specific training exercise. This will allow the evaluator to effectively evaluate whether the user is focusing his or her vision in an optimal manner during a simulation of real-world conditions. For instance, a football coach may see exactly where a quarterback is focusing his vision throughout the running of a simulated football play during practice. Thus, the coach can see exactly which receiver the quarterback is focused on at any given time during the play, as well as how the quarterback is progressively scanning his receiver options as a play progresses. Because the display screen 702 shows the location of the point of gaze 401 continuously as the play progresses, the coach may also be able to evaluate how quickly the quarterback is able to scan the field from player to player to locate open receivers and how quickly the quarterback is able to throw the ball once an open receiver is located, as timing is a critical factor in distributing the football effectively.

In carrying out the present method, a first set of data may be generated representing the point of gaze of a user 101 in real time. The data may then be processed into a graphical representation 401 of the point of gaze of the user 101 in real time, and the graphical representation 401 of the point of gaze may then be displayed in real time superimposed onto streaming video data captured by the scene camera 104 in real time.

Generating the data representing the point of gaze of the user 101 in real time comprises the user 101 donning headgear 105 having an optional first optical sensor 102, a second optical sensor 103, and a scene camera 104 secured to the headgear. In a preferred embodiment, the system includes the first sensor 102, which is preferably a contact lens fitted to at least one of the user's eyes. The first sensor 102 is preferably configured to generate a first stream of data representing the point of gaze of the user 101 by means of a thin-film camera 305, as well as by creating a magnetic field via magnetic material 502 embedded within the first sensor 102. The first sensor 102 is preferably further configured to transmit the first set of data to a data processing unit 701, which may preferably also be secured to the headgear. The second optical sensor 103 may be utilized independently for tracking the eye movements of the user, or the second sensor 103 may preferably be used in conjunction with the first sensor 102. The second sensor 103 may preferably be configured to generate a second stream of data representing the point of gaze of the user 101 by gathering data in the form of infrared corneal reflections. In a preferred embodiment, the second sensor 103 may additionally gather data in the form of magnetic field polarization adjustments. The second sensor 103 may be further configured to transmit the second set of data to the data processing unit 701. The scene camera 104 is configured to capture an area of the user's field of view by recording a stream of video data capturing at least an area of the central and peripheral areas of view of the user 101. The scene camera is configured to transmit the stream of video data to the data processing unit 701.

The above-referenced data representing the point of gaze of the user 101 in real time may then be processed. The data processing unit 701 may receive the first stream of data and the second stream of data, as well as the stream of video data. The data processing unit 701 then processes the first stream of data, the second stream of data, and the stream of video data into a synchronized stream of data. This synchronized stream of data serves as a singular representation of all forms of input data serving the same purpose, which is to determine a location where the user 101 is looking. The data processing unit 701 then processes the synchronized stream of data into a graphical representation 401 of the point of gaze of the user 101 in real time. This allows for the POG to be seen by the viewer and applied to the setting of the user 101. The data processing unit 701 may then encode the graphical representation 401 through the encoder 902 as a preparation means for transmitting the data. The data processing unit 701 may then transmit the graphical representation 401 to a server 901. Finally, displaying the graphical representation 401 may comprise a server 901 transmitting the graphical representation 401 of the point of gaze of the user 101 in real time to an image viewer 702, wherein the server 901 preferably comprises a dedicated media server 901.

It is understood that versions of the invention may come in different forms and embodiments. Additionally, it is understood that one of skill in the art would appreciate these various forms and embodiments as falling within the scope of the invention as disclosed herein.

What is claimed is:

1. An athletic training method comprising the steps of:
providing a headgear;
donning the headgear, by an athlete, and securing the headgear in a fixed position on the athlete's head;
providing a gaze-tracking system comprising:
an optical sensor secured to the headgear and adapted to track eye movement, wherein the optical sensor is positioned to track eye movements of the athlete when the athlete is donning the headgear,
a scene camera secured to the headgear, wherein the scene camera is positioned facing in a forward direction from the athlete's head and configured to capture an area of a field of view of the athlete when the athlete is donning the headgear,
a display screen configured to display video data generated by the scene camera, and
a data processing unit in communication with the optical sensor, the scene camera, and the display screen,
wherein the system is configured to continuously determine a point of gaze of the athlete based on the eye movements of the athlete and to continuously display, on the display screen, the point of gaze superimposed onto streaming video data captured by the scene camera in real time;
running an athletic training play, by at least the athlete donning the headgear;
recording video of the field of view of the athlete, using the scene camera;
simultaneously tracking eye movements of the athlete in real time, by the optical sensor, while running the athletic training play; and
graphically displaying, on the display screen, the point of gaze superimposed onto the recorded video over a period of time in which the gaze-tracking system is activated while running the athletic training play.

2. The method of claim 1, wherein the gaze-tracking system further comprises a contact lens configured to fit onto an eye of the athlete, wherein the contact lens comprises a contact lens camera and a transmitter configured to communicate with the optical sensor, wherein the step of tracking eye movements comprises tracking eye movements by both the optical sensor and the contact lens.

3. The method of claim 2, wherein the contact lens comprises magnetic material embedded within contact lens, wherein the optical sensor further comprises a magnetic sensor configured to measure polarization of a magnetic field generated by the magnetic material.

4. The method of claim 2, wherein the contact lens camera is capable of recording a series of thin-film images.

5. The method of claim 1, wherein the optical sensor comprises a vector camera configured to record infrared light quantities in real time, wherein the step of tracking eye movements comprises the optical sensor measuring infrared corneal light reflections from an eye of the user.

6. The method of claim 1, wherein the headgear is a football helmet.

7. A training method comprising the steps of:
providing a headgear;
donning the headgear, by a user, and securing the headgear in a fixed position on the user's head;
providing a gaze-tracking system comprising:
an optical sensor secured to the headgear and adapted to track eye movement, wherein the optical sensor is positioned to track eye movements of the athlete when the athlete is donning the headgear,
a scene camera secured to the headgear, wherein the scene camera is positioned facing in a forward direction from the user's head and configured to capture an area of a field of view of the user when the user is donning the headgear,
a display screen configured to display video data generated by the scene camera, and
a data processing unit in communication with the optical sensor, the scene camera, and the display screen,
wherein the system is configured to continuously determine a point of gaze of the user based on the eye movements of the user and to continuously display, on the display screen, the point of gaze superimposed onto streaming video data captured by the scene camera in real time;
physically simulating real-world conditions, by the user donning the headgear;
recording video of the field of view of the user, using the scene camera;
simultaneously tracking eye movements of the user in real time, by the optical sensor, while the user is simulating real-world conditions; and
graphically displaying, on the display screen, the point of gaze superimposed onto the recorded video over a period of time in which the gaze-tracking system is activated while the user is simulating real-world conditions.

8. The method of claim 7, wherein the gaze-tracking system further comprises a contact lens configured to fit onto an eye of the user, wherein the contact lens comprises a contact lens camera and a transmitter configured to communicate with the optical sensor, wherein the step of tracking eye movements comprises tracking eye movements by both the optical sensor and the contact lens.

9. The method of claim 8, wherein the contact lens comprises magnetic material embedded within contact lens, wherein the optical sensor further comprises a magnetic sensor configured to measure polarization of a magnetic field generated by the magnetic material.

10. The method of claim 8, wherein the contact lens camera is capable of recording a series of thin-film images.

11. The method of claim 7, wherein the optical sensor comprises a vector camera configured to record infrared light quantities in real time, wherein the step of tracking eye movements comprises the optical sensor measuring infrared corneal light reflections from an eye of the user.

12. The method of claim 7, wherein the headgear is a football helmet.

13. A gaze-tracking system comprising:
a headgear configured to secure to a user's head in a fixed position relative to the user's head;
an optical sensor secured to the headgear and adapted to track eye movement, wherein the optical sensor is positioned to track eye movements of the user when the user is donning the headgear;
a scene camera secured to the headgear, wherein the scene camera is positioned facing in a forward direction from the user's head and configured to capture an area of a field of view of the user when the user is donning the headgear;
a display screen configured to display video data generated by the scene camera, and
a data processing unit in communication with the optical sensor, the scene camera, and the display screen,
wherein the system is configured to continuously determine a point of gaze of the user based on the eye movements of the user tracked by the optical sensor and to continuously display, on the display screen, the point of gaze superimposed onto streaming video data captured by the scene camera in real time.

14. The gaze-tracking system of claim 13, wherein the gaze-tracking system further comprises a contact lens configured to fit onto an eye of the user, wherein the contact lens comprises a contact lens camera and a transmitter configured to communicate with the optical sensor.

15. The gaze-tracking system of claim 14, wherein the contact lens comprises magnetic material embedded within contact lens, wherein the optical sensor further comprises a magnetic sensor configured to measure polarization of a magnetic field generated by the magnetic material.

16. The gaze-tracking system of claim 14, wherein the contact lens camera is capable of recording a series of thin-film images.

17. The gaze-tracking system of claim 13, wherein the optical sensor comprises a vector camera configured to record infrared light quantities in real time.

18. The gaze-tracking system of claim 13, wherein the headgear is a football helmet.
